(19) United States
(12) Patent Application Publication
(10) Pub. No.: US 2008/0227081 A1
(43) Pub. Date: Sep. 18, 2008

(54) PROCESS FOR DECOMPOSING HYDROGEN SULFIDE BY MEANS OF FEEDING OXYGEN

(76) Inventor: Christoph MARTENS, Rockstedt (DE)

Correspondence Address: MCGLEW & TUTTLE, PC, P.O. BOX 9227, SCARBOROUGH STATION, SCARBOROUGH, NY, 10510-9227, US

(21) Appl. No.: 12/048,359

(22) Filed: Mar. 14, 2008

(30) Foreign Application Priority Data
Mar. 15, 2007 (DE) 10 2007 013 190.0

(51) Int. Cl. C12Q 3/00; C12P 3/00; C12M 1/36; C12M 1/04

(52) U.S. Cl. 435/3; 435/168; 435/289.1; 435/286.6

(57) ABSTRACT

A process is provided for decomposing hydrogen sulfide in a biogas fermenter (10) for generating biogas from biomass, which is fed as a fermentation substrate into the biogas fermenter (10). A reaction gas with increased oxygen content compared to air is fed into a gas space (26) of the biogas fermenter (10).

### CROSS REFERENCE TO RELATED APPLICATIONS

This application claims the benefit of priority under 35 U.S.C. § 119 of German Patent Application 10 2007 013 190.0 filed Mar. 15, 2007, the entire contents of which are incorporated herein by reference.

### FIELD OF THE INVENTION

The present invention pertains to a process for decomposing hydrogen sulfide in a biogas fermenter to generate biogas from biomass, which is fed as a fermentation substrate to the biogas fermenter.

### BACKGROUND OF THE INVENTION

DE 10 2005 041 798 A1 discloses a process for decomposing hydrogen sulfide in a biogas fermenter to generate biogas. Biomass is fed as a fermentation substrate to the biogas fermenter.

Hydrogen sulfide is formed during the production of biogas. Sulfur is an indispensable element for the nutrition of organisms and it therefore inevitably enters the fermenting reactor of biogas plants with the substrate in the form of sulfates and sulfides. Based on the anaerobic decomposition processes, hydrogen sulfide is formed by the partial transformation or the bound sulfur compounds. A considerable quantity of gaseous hydrogen sulfide is formed in the fermenting tank especially during the fermentation of substances with a high protein content.

Toxic sulfur dioxide is formed from the hydrogen sulfide during the combustion of the biogas. Furthermore, hydrogen sulfide and sulfur dioxide damage, for example, fittings or motor components due to corrosion and may contribute to the accelerated acidification of motor oil. This causes high repair and maintenance costs and therefore justifies the lasting need for effective and inexpensive biogas desulfurization.

The desulfurization of biogas will be of increasing significance in the future. Thus, the biogas must be freed of hydrogen sulfide before it is used in fuel cells, because hydrogen sulfide directly affects important components of such fuel cells. Furthermore, the percentage of methane in the biogas must be increased in order to make it possible to feed the biogas into the existing natural gas pipeline networks. It is also especially necessary for this to raise the percentage of methane in the biogas to the level present in natural gas, because the calorific value of the mixed gas would otherwise decrease. It is therefore necessary to free the biogas from carbon dioxide, for example, via a gas scrubber or filtration. This is carried out, for example, via an amine scrubber, which requires a biogas that is nearly completely free from hydrogen sulfide. Not only can the biogas thus processed to natural gas quality be fed into the natural gas pipeline network, but it can also be used to fill the tanks of natural gas-powered vehicles at corresponding filling stations.

It is known that air can be fed into a biogas fermenter. Oxygen, a component of air, is used to supply hydrogen sulfide-decomposing bacteria. The hydrogen sulfide is transformed by the bacteria either into pure sulfur and water or into sulfurous acid.

It is disadvantageous in this prior-art desulfurization process that not only the oxygen needed for the transformation of the hydrogen sulfide, which oxygen is present at a concentration of about 21%, is fed into the gas space by blowing in air. With a percentage of about 79%, nitrogen, which is not needed, is also fed in with the feeding of air. It becomes a part of the biogas and lowers the calorific value and hence the quality of the biogas. Even though it is mentioned in paragraph [0005] in DE 10 2005 041 798 A1 that the hydrogen sulfide is transformed into elemental sulfur when oxygen is fed into the gas space of the fermenter, the oxygen in this context is ultimately only meant as a component of the air as the gas transformed by the microorganisms decomposing hydrogen sulfide. Since the oxygen itself is disturbing in the anaerobic fermentation process in the fermenter, namely, even toxic for the microorganisms producing the methane gas, experts have always sought to introduce as little oxygen as possible into the fermenter. This is also documented by DE 199 38 853 A1. This document pertains to a process in which an anaerobic fermentation process is carried out first and an aerobic composting process is carried out thereafter. Either two separate tanks are made available for this, or the process is carried out in one and the same tank consecutively in time. An air separation means, by which the air is separated into its principal constituents, namely, nitrogen and oxygen, is associated with the plant. The nitrogen thus obtained is fed into the process during the anaerobic process, while the oxygen obtained is fed in during the aerobic composting process. The reason for the separation is that the oxygen is disturbing during the anaerobic fermentation process in this case as well.

Furthermore, a biogas plant is known from DE 10 2004 035 997 A1; it is specifically a wood gasifying apparatus, in which a pyrolysis process takes place. In a concrete exemplary embodiment in this state of the art, processed air with an increased percentage of oxygen is made available for the pyrolysis. However, this oxygen is not used for the desulfurization process, but purely for the pyrolysis. As is stated elsewhere in this document, the desulfurization is carried out by blowing in air.

Furthermore, a device for removing sulfur-containing compounds from biogas is known from AT 007985 U1. The sulfur compounds are decomposed according to AT 007985 U1 in an absorber arranged downstream of the biogas reactor proper by means of oxygen. The absorber arranged downstream of the biogas fermenter proper represents an additional plant component with additional costs.

Finally, DE 197 44 653 A1 discloses a biogas plant, in which desulfurization is likewise carried out by feeding in oxygen. The biogas obtained in the biogas reactor proper is stored intermediately in a separate gas dome, in which the oxygen is fed in. As a result, there is a certain separation in space between the microorganisms producing the methane gas in the fermentation substrate and the microorganisms in the gas dome, which react the oxygen and decompose the hydrogen sulfide. However, the oxygen is mentioned ultimately only as a component of the air in this document as well.

### SUMMARY OF THE INVENTION

The basic object of the present invention is to preventively prevent or reduce the percentage of nitrogen in the biogas and thus to improve the quality of the biogas produced by means of a biogas fermenter.

To accomplish the object, the process according to the present invention is characterized in that a reaction gas with an oxygen content that is higher than that in air is fed into a gas space of the biogas fermenter.

The decomposition of hydrogen sulfide in the biogas is facilitated by the measure according to the present invention in a surprisingly simple manner without additionally introducing a needlessly large quantity of nitrogen into the biogas. This is surprising above all because the fermentation process for producing the biogas takes place as an anaerobic process; however, a gas with increased oxygen content is fed directly into biogas in the fermenter according to the present invention. Furthermore, a smaller gas volume, which must be fed into the fermenting space, is needed compared to the use of air. Due to the higher oxygen content, a smaller gas volume is sufficient to transform an equal quantity of hydrogen sulfide and to reduce the quantity of nitrogen introduced, as a result of which the percentage of nitrogen in the biogas is also preventively reduced. The percentage of methane in the biogas increases, as a result of which the quality of the biogas is improved. In addition, the increased percentage of oxygen in the gas fed in can lead to a substantial optimization of desulfurization when only a part of the quantity of gas fed in may possibly be available for the desulfurizing microorganisms in areas that are difficult to reach.

According to a variant of the present invention, the quantity of hydrogen sulfide and/or nitrogen and/or oxygen in the biogas is measured at an outlet for the biogas from the biogas fermenter, and the quantity of reaction gas fed in is controlled as a function of the measured values. It is ensured hereby that only the quantity of reaction gas that is necessary for the decomposition of the hydrogen sulfide is always fed into the biogas fermenter. If the percentage of hydrogen sulfide is too high, the reaction gas feed is increased, and if the percentage of oxygen is too high, the feed of reaction gas is reduced. It is ensured hereby that the oxygen introduced into the biogas fermenter will be consumed by the hydrogen sulfide-decomposing microorganisms as completely as possible and cannot enter the fermentation substrate, where it would be toxic for the microorganisms producing methane gas.

The above-mentioned effect is further improved by the fact that the reaction gas is fed by means of feed lines as directly as possible to surfaces on which microorganisms decomposing hydrogen sulfide grow. The oxygen fed in, in the reaction gas, can thus be absorbed directly by these microorganisms and cannot enter the fermentation substrate.

According to a variant of the present invention, the gas can be generated with increased oxygen content by enriching air with oxygen. In particular, the gas can be produced only immediately before being fed in by enriching the air with oxygen. The gas to be fed in can consequently be produced directly at the plant. Production of the gas at another site and the transportation to the plant and storage, which may be complicated and expensive, are thus not necessary.

Furthermore, it is advantageous to feed oxygen of industrial purity. The quantity of gas injected can be reduced as a result by up to 79% in the biological desulfurization of air. The effectiveness of the desulfurization of air is thus increased even more. The disturbing percentage of nitrogen is not introduced into the gas space at all.

Corresponding to an advantageous embodiment of the process according to the present invention, an oxygen feed unit for feeding a gas with increased oxygen content compared to air is associated with the biogas fermenter.

The present invention will be explained below on the basis of an exemplary embodiment by means of drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic side view showing a first embodiment of a device with the features of the present invention; and

FIG. 2 is a schematic side view another exemplary embodiment of a device with the features of the present invention.

### DESCRIPTION OF THE PREFERRED EMBODIMENTS

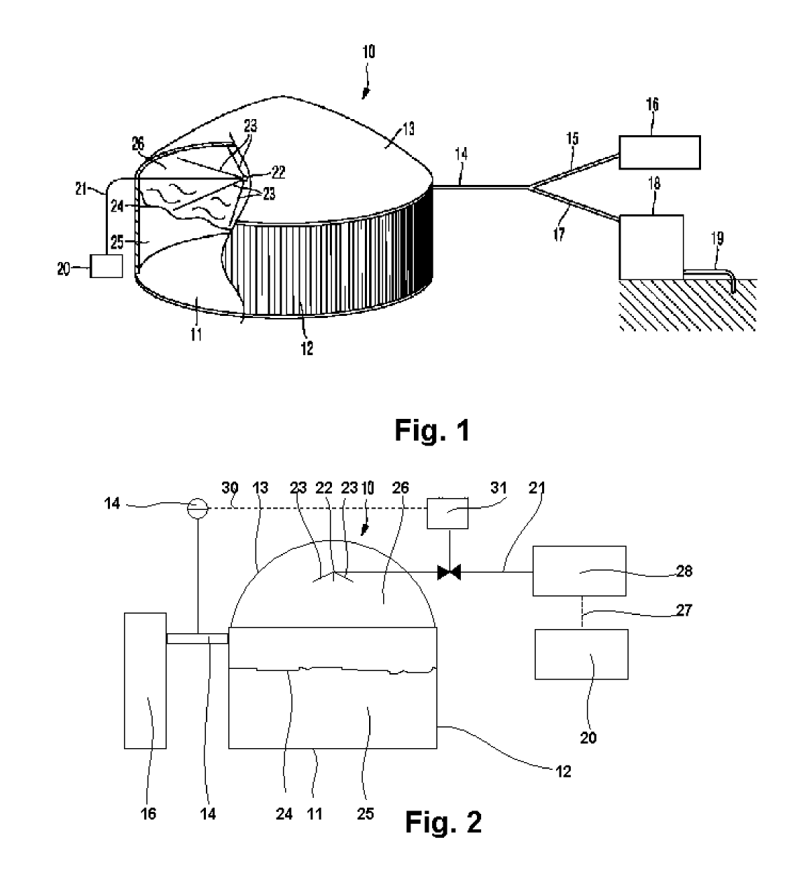

Referring to the drawings in particular, FIG. 1 shows a schematic view of a biogas fermenter 10. The biogas fermenter 10 has a circular bottom plate 11 and a circumferential tank wall 12. An air-supported film cover 13 is used to cover the biogas fermenter 10. A fermentation substrate 25, from which a biogas is formed by anaerobic fermentation, is contained in the biogas fermenter 10. The biogas enters a gas space 26 via a fermentation substrate surface 24. The gas space 26 is above the fermentation substrate 25 in the biogas fermenter 10. The biogas is removed from the gas space 26 of the biogas fermenter 10 via a gas line 14. The biogas can be fed directly into a user 16, for example, a block-type thermal power plant, via a branch line 15 branched off from the gas line 14. The biogas can be fed, additionally or alternatively, to a gas purifier 18 via another branch line 17 branched off from the gas line 14.

The percentage of methane in the biogas is raised to the level present in natural gas by means of the gas purifier 18. The gas purification 18 can free the biogas from carbon dioxide by means of a gas scrubber or filtration. The biogas prepared by the gas purifier 18 is subsequently fed into the natural gas pipeline network 19. The gas purification 18 may be carried out, for example, by means of an amine scrubber. However, this requires a biogas that is freed nearly completely from hydrogen sulfide (H₂S), which is always formed during the fermentation process.

One possibility of decomposing hydrogen sulfide is offered by corresponding bacteria, which transform the hydrogen sulfide into sulfur and water (2H₂S+O₂-->2S+2H₂O) or sulfurous acid (2H₂S+3O₂-->2H₂SO₃) while taking up oxygen. It is necessary for this to supply the hydrogen sulfide-decomposing bacteria in the biogas fermenter 10 with oxygen.

Ideal living conditions prevail in the gas space 26 per se above the fermentation substrate surface 24 for the hydrogen sulfide-decomposing bacteria, with the exception of the missing oxygen. Oxygen at this site could, however, enter the fermentation substrate 25, which is being kept steadily in motion by means of a stirrer (not shown) and interfere with the anaerobic fermentation process in the fermentation substrate 25. This is, however, actually not the case.

A gas with an increased oxygen content compared to that in air, concretely oxygen of industrial purity, is fed into the gas space 26 of the biogas fermenter 10. An oxygen generator 20, which produces oxygen of industrial purity from ambient air, is associated for this purpose with the biogas fermenter 10. This oxygen is consequently produced directly at the plant and is fed via a feed line 21 to a distributor 22. The distributor 22 distributes the gas uniformly via distribution lines 23 and over the fermentation substrate surface 24. The distribution lines 23 are designed concretely such that they feed the oxygen directly to surfaces on which hydrogen sulfide-decomposing microorganisms grow, so that the oxygen fed in can be taken up possibly directly by these microorganisms.

The oxygen generated in the oxygen generator 20 is first fed via a line 27 into a storage means 28 and stored there intermediately in the exemplary embodiment shown in FIG. 2. A sufficient quantity of oxygen is always available as a result.

Furthermore, the composition of the biogas is measured at a measuring point 29 on the gas line 14, through which the biogas is removed from the biogas fermenter 10. Concretely, the quantities of hydrogen sulfide, nitrogen and/or oxygen contained in the biogas are determined and made available to a control device 31 via a data line 30. This control device 31 controls the quantity of oxygen fed into the gas space 36 via a valve 32 as a function of the measured values. If the hydrogen sulfide content in the gas line 14 is too high, i.e., the hydrogen sulfide is not decomposed sufficiently completely, the quantity of oxygen is increased. If, by contrast, no or hardly any hydrogen sulfide is contained, but the quantity of oxygen is too high, the quantity of oxygen fed in is reduced. If, as was indicated above, the quantity of nitrogen in the biogas is also determined in the gas line 14, this can be controlled for controlling the separation of the air into oxygen and nitrogen in the oxygen generator 20, should a certain quantity of nitrogen in the biogas nevertheless be desirable for certain applications.

The gas volume introduced into the biogas fermenter 10 can be reduced by feeding in the oxygen made available by means of the oxygen generator 20, because the gas contains an increased percentage of oxygen compared to the use of air. At the same time, the percentage of unnecessary nitrogen can be reduced to a minimum, especially when oxygen of industrial purity is used.

While specific embodiments of the invention have been shown and described in detail to illustrate the application of the principles of the invention, it will be understood that the invention may be embodied otherwise without departing from such principles.

What is claimed is:

1. A process for decomposing hydrogen sulfide in a biogas fermenter for generating biogas from biomass, the process comprising the steps of:
feeding biomass into the biogas fermenter as a fermentation substrate;
feeding a reaction gas with increased oxygen content compared to air into a gas space of the biogas fermenter.

2. A process in accordance with claim 1, further comprising:
measuring a quantity of hydrogen sulfide and/or nitrogen and/or oxygen in the biogas measured at an outlet for the biogas from the biogas fermenter to provide a measured value of hydrogen sulfide and/or nitrogen and/or oxygen; and
controlling a quantity of reaction gas fed into the gas space of the biogas fermenter as a function of the measured value.

3. A process in accordance with claim 1, wherein the reaction gas is fed to surfaces on which microorganisms decomposing hydrogen sulfide grow by means of said feed lines.

4. A process in accordance with claim 1, wherein the gas with increased oxygen content is produced by enriching air.

5. A process in accordance with claim 4, wherein the gas with increased oxygen content compared to air is produced immediately before being fed into the biogas fermenter.

6. A process in accordance with claim 1, wherein oxygen of industrial purity is fed into the biogas fermenter as the reaction gas.

7. A process for decomposing hydrogen sulfide in a biogas fermenter for generating biogas from biomass, the process comprising the steps of:
feeding biomass into the biogas fermenter as a fermentation substrate to form a body of fermentation substrate with a gas space and with a fermentation substrate surface at a fermentation substrate gas space interface;
feeding a reaction gas with a higher oxygen content than air into the gas space of the biogas fermenter.

8. A process in accordance with claim 7, further comprising:
measuring a quantity of hydrogen sulfide and/or nitrogen and/or oxygen in the biogas measured at an outlet for the biogas from the biogas fermenter to provide a measured value of hydrogen sulfide and/or nitrogen and/or oxygen; and
controlling a quantity of reaction gas fed into the gas space of the biogas fermenter as a function of the measured value.

9. A process in accordance with claim 8, wherein the reaction gas is fed and directed by feed lines toward said fermentation substrate surface and microorganisms decomposing hydrogen sulfide grow on said fermentation substrate surface.

10. A process in accordance with claim 9, wherein the reaction gas is produced by enriching air.

11. A process in accordance with claim 10, wherein the reaction gas is produced immediately before being fed into the biogas fermenter.

12. A process in accordance with claim 11, wherein the reaction gas has an oxygen content of industrial purity oxygen.

13. A system for decomposing hydrogen sulfide in a biogas fermenter for generating biogas from biomass, the system comprising:
a biogas fermenter;
a biomass feed for feeding biomass into the biogas fermenter as a fermentation substrate to form a body of fermentation substrate with a gas space and with a fermentation substrate surface at a fermentation substrate gas space interface;
a reaction gas feed for feeding reaction gas with a higher oxygen content than air into the gas space of the biogas fermenter.

14. A system in accordance with claim 13, further comprising:
a biogas outlet feed for extracting biogas from the biogas fermenter;
a measurement means for measuring a quantity of hydrogen sulfide and/or nitrogen and/or oxygen in the biogas at said outlet for the biogas from the biogas fermenter to provide a measured value of hydrogen sulfide and/or nitrogen and/or oxygen; and
a controller for controlling a quantity of reaction gas fed into the gas space of the biogas fermenter as a function of the measured value.

15. A system in accordance with claim 14, wherein said reaction gas feed includes feed lines for directing reaction gas toward said fermentation substrate surface for promoting microorganisms decomposing hydrogen sulfide to grow on said fermentation substrate surface.

16. A system in accordance with claim 15, further comprising an oxygen generator for producing the reaction gas by enriching air.

17. A system in accordance with claim 16, wherein said oxygen generator is positioned adjacent to said biogas fermenter for producing said he reaction gas immediately before being fed into the biogas fermenter.

18. A system in accordance with claim 17, wherein the reaction gas produced by said oxygen generator has an oxygen content of industrial purity oxygen.
